(19) United States
(12) Patent Application Publication
(10) Pub. No.: US 2006/0229433 A1
(43) Pub. Date: Oct. 12, 2006

(54) Modified antiviral peptides with increased activity and cell membrane affinity

(75) Inventors: Bonabes O. De Rouge, Paris (FR); Kamel Mabrouk, Les Pennes Mirabeau (FR); Jean-Marc Sabatier, Rousset (FR)

Correspondence Address: DARBY & DARBY P.C., P. O. BOX 5257, NEW YORK, NY, 10150-5257, US

(73) Assignee: Cellpep SA, Paris (FR)

(21) Appl. No.: 10/557,583

(22) PCT Filed: May 20, 2004
(86) PCT No.: PCT/EP04/05563

§ 371 Date: Mar. 3, 2006

(30) Foreign Application Priority Data
May 20, 2003 (GB) 0311565.6
Aug. 20, 2003 (GB) 0319514.6

(51) Int. Cl. C07K 7/08; C07K 7/06

(52) U.S. Cl. 530/329; 530/330

(57) ABSTRACT

The activity and cell membrane affinity of certain antiviral multiple branch peptide constructions, including those known from WO 95/07929, WO 98/29443 and WO 03/95479, can be improved by bonding to the C-end of the peptide a terminator which is either (a) an ω-amino-fatty acid having from 4 to 10 carbon atoms and from 0 to 2 carbon-carbon double bonds or (b) a peptidic cell membrane penetrating agent. The improvement is so marked that in some cases the number of branches can be reduced, sometimes to a single branch, and/or that the branches may be shortened. The preferred ω-amino-fatty acids are γ-aminobutyric acid, δ-aminovaleric acid and ε-aminocaproic acid. The peptidic cell membrane penetrating agent is suitably a TAT-derived peptide, penetratin® or Kpam.

### CROSS-REFERENCE TO PRIOR APPLICATIONS

This is a U.S. national phase application under 35 U.S.C. §371 of International Patent Application No. PCT/EP2004/005563 filed on May 20, 2004, and claiming priority to British Application 0311565.6 filed May 20, 2003 and British Application 0319514.6 filed Aug. 20, 2003, all of which are incorporated by reference herein in their entirety. The International Application was published in English on Dec. 2, 2004 as WO 2004/104031 A2 under PCT Article 21(2).

### DESCRIPTION

The invention relates to compounds with increased antiviral activity, in particular increased anti-HIV activity, due to the covalent graft on the original antiviral molecule of a structure capable of cell membrane interaction and/or crossing.

### BACKGROUND

Multiple branch peptide contstructions (MBPCs) comprise a core matrix to which small peptides are bonded. The core matrix is a dendritic polymer which is branched in nature, preferably with each of the branches thereof being identical. Although other core molecules are possible, the preferred core molecule is lysine. The core matrix can be built up from a central lysine residue, sometimes called the root of the MBPC. Two lysine residues are bonded to the central lysine residue, each through its carboxyl group to a different one of the amino groups of the central lysine residue. This provides a molecule with four amino groups, which may be the core matrix for an MBPC having four peptides. Alternatively by bonding a further four lysine residues, each through its carboxyl group to a different one of the said four amino groups, one can provide a molecule with eight branches. This molecule can serve as the core matrix for an MBPC having eight peptides or can alternatively receive eight lysine residues in the manner described above to form a core matrix for an MBPC having sixteen peptides. The C-ends of peptides are covalently bonded to each of the branches of the core matrix to form the MBPC. The peptides may be the same, which is preferred, or may be different from one another. The resulting molecule has a cluster of peptides at the surface and an interior core matrix which is not presented and is therefore not antigenic.

Spacers may, if desired, be included between the peptides and the core matrix. The carboxyl group of the first lysine residue may be left free, amidated, or coupled to a blocking compound such as β-alanine (β-aminopropionic acid). Peptides can include D or L-amino acid residues. D amino acids last longer in vivo because they are harder for peptidase to cut, but the L amino acids have better activity. Moreover, peptide analogues, synthetic constructs using the carbon skeleton of peptides but omitting the —CONH— peptide bonds, can be employed in place of peptides. Thus, it should be understood that references to peptides herein may also be taken to include peptide analogues. It is believed that peptide analogues will be more resistant to peptidase and last longer in vivo. If the peptide is too long, the MBPC will become antigenic. It is therefore desirable that each peptide should have not more than ten, and preferably not more than nine, amino acid residues.

MBPCs for use in the treatment of HIV infections were first described by J-M. Sabatier et al in WO 95/07929. The MBPCs described therein have peptides which contain the sequence GPGR (from the V3 loop of the surface envelope glycoprotein gp120 of HIV) preceded by from 0 to 4 amino acid residues and succeeded by from 2 to 4 amino acid residues. The amino acid sequences IGPGR and IXXGPGR (where X is an amino acid residue) are excluded. The most preferred of these MBPCs has a lysine residue core with eight peptides GPGRAF bonded thereto. It may be represented as (GPGRAF)₈-K₄-K₂-K-βA-OH, the OH terminal indicating the carboxyl group of the β-alanine. That carboxyl group may alternatively be modified to form a carboxamide terminal. This compound is referred to herein as SPC3.

In WO 98/29443, J-M Sabatier et al described further MBPCs which may be effective in the treatment of HIV infection. These use peptides derived from the HIV envelope transmembrane glycoprotein gp41. The peptides contain the sequence RQGY preceded by from 0 to 4 amino acid residues and succeeded by from 2 to 4 amino acid residues. The most preferred of these MBPCs has a lysine residue core with eight peptides RQGYSPL bonded thereto. It may be represented as (RQGYSPL)₈-K₄-K₂-K-βA-OH, the OH terminal indicating the carboxyl group of the β-alanine. That carboxyl group may alternatively be modified to form a carboxamide terminal. This compound is referred to herein as RL, although it has in the past also been referred to as SPC RL and as RL41.

Subsequently to WO 98/29443, it was established that the MBPC (RQGYSPL)₂-K-βA (hereinafter RL dimer) is effective but that the MBPC (RQGYSP)₂-K-βA is less so. This was thought to confirm the lower limit of 6 amino acids in the peptide branches of the MBPCs. However, K Mabrouk et al showed in WO 03/095479 that some shorter peptides could be used, in particular (RQGYS)₂-K-βA-OH (hereinafter RS, but in the past also referred to as Short RL) and (RQGY)₈-K₄-K₂-K-βA-OH.

SPC3 and RL both have 8 branches and are described as octomers. RS has two branches, and is described as a dimer. None of the monomers, that is the linear peptides GPGRAF, RQGYSPL and RQGYS, has ever shown any activity.

Anti HIV agents such as SPC3 and RL have been shown to block the fusion step of retroviral infection through direct interaction with cell membrane receptors; other anti fusion agents such as enfuvirtide and T-1249 (Trimeris Inc) interact directly with the viral envelope glycoproteins. The activity of the latter depends on the structure of such glycoproteins, and therefore on the viral strain. Ultimately, molecules that interfere directly with viral glycoproteins will lead to the selection of resistant strains. On the contrary, molecules which are able to block cell membrane receptors should not lead to viral selection, as all strains will be similarly inhibited.

Cell receptor blocking HIV inhibitors may interact with the surface of such receptors (for instance CxCR4 or CCR5) but also with intra membrane components of said receptors, or even with sub-membrane sites or events.

As an example, SPC3, which is an extremely water-soluble peptide, has an anti HIV activity in vitro on C8166 cultured cells as well as on peripheral blood lymphocytes (PBL) and on macrophages. B de Rougé in WO 99/34777 showed that this activity is increased 5 to 50 times when SPC3 is associated with certain types of liposomes, probably because of better interaction with cell membranes. However, SPC3 is a polymerized peptide of 56 amino-acid residues. Its association with liposomes is difficult and the yield is not perfect, leading to cost increases as well as technical risks. Other means of improving the efficacy of molecules like SPC3 have therefore been sought.

### The Invention

The invention provides a compound comprising a water soluble antiviral peptide including one of the sequences GPG and RQGY and, bonded to the C-end of the peptide, a terminator which is either (a) an ω-amino-fatty acid having from 4 to 10 carbon atoms and from 0 to 2 carbon-carbon double bonds or (b) a peptidic cell membrane penetrating agent.

The antiviral peptide may be an MBPC with a lysine core matrix. In such a case the terminator is bonded to the root lysine residue. The MBPCs described above may be used, that is to say SPC3 which has 8 branches of GPGRAF, RL which has 8 branches of RQGYSPL and RS which has 2 branches of RQGYS. However, the improvement resulting from the bonding of the terminator to the C-end of the antiviral peptide is so great that SPC3 and RL can be reduced to two branches (SPC3 dimer and RL dimer, respectively), or even to one branch (SPC3 monomer and RL monomer, respectively), while RS may also be reduced to one branch (RS momomer). Further work has even indicated that SPC3 monomer (GPGRAF) may be shortened to GRGRA, GPGR or GPC. As these are much smaller molecules, they are much easier and cheaper to make and are preferred for that reason.

The ω-amino-fatty acid is preferably saturated. Longer chains than 10 carbon atoms are unnecessary as the effect is obtained with less, and longer chains may be too lipidic. The preferred length is from 4 to 8 carbon atoms, and more preferably from 4 to 6 carbon atoms. The most preferred ω-amino-fatty acids are γ-aminobutyric acid, δ-aminovaleric acid and ε-aminocaproic acid.

The peptidic cell membrane penetrating agent is suitably a TAT-derived peptide, penetratin® or Kpam, although other peptides may also be suitable.

### EXPERIMENTAL

We first synthesized SPC3 octomers, with the graft of saturated fatty acid chains of increasing length, from 4 to 8 carbons, on the core lysine residue; and SPC3 octomers with three different peptide chains on the lysine residue: a TAT-derived peptide, penetratin, and Kpam peptide, all reported to enhance membrane penetration and crossing. We tested the above molecules on C8166 cells infected with NL 4-3 HIV strain, then on PBL with the same strain. Results are shown in Tables 1 and 2.

When positive results were observed, further attempts were made to test whether the graft of membrane affinity chains on the water soluble peptides could allow for a reduction in their size without losing efficacy (SPC3, RL and their derivatives are polymers, often octomers, of small peptides; the monomers have been shown to be inactive), with a view of cost-containment. To this end we synthesized monomers and dimers of the sequences of SPC3, RL and RS, with the addition of the preferred grafted sequence, and tested them on C8166, PBL and PBMC. Results are shown in Tables 3 to 5.

We also synthesized shortened peptides related to SPC3 monomer, which is GPGRAF, in particular GRGRA, GPGR and GPG and tested these with a 6-aminovaleric acid terminator. These were tested twice, 8 days apart, on C8166 cells against HIV-1 NL 4-3 (results are shown in Tables 6 and 7) and on C8166 cells against HIV-1 NDK (results are shown in Table 8).

Whilst the experiments conducted so far are in vitro, it is expected that the modifications made in this invention will lead to better availability of the compounds in the lymphatic system in vivo.

Test Methods

Cells and Viruses.

HIV-1 NL 4-3 isolate (Adachi et al., 1986; Barré-Sinoussi et al., 1983) and highly cytopathic Zairian HIV-1 NDK isolate (Ellrodt et al., 1984) was propagated in permissive CEM cells (Nara et al., 1987). Uninfected CEM and C8166 (Salahuddin et al., 1983) were maintained in RPMI 1640 (R10) with ultraglutamine (cambrex, Vervier, Belgium), penicillin (100 U/ml), streptomycin (100 μg/ml), and 10% heat-inactivated fetal calf serum (Cambrex).

Peripheral blood lymphocytes from an HIV-1 negative donor were grown as described earlier, maintained in RPMI 1640 with ultraglutamine, supplemented with IL2 (20 μg/ml), penicillin (100 U/ml), streptomycin (100 μg/ml), and 10% heat-inactivated fetal calf serum. Cells were stimulated three days in the medium supplemented with phytohemagglutinin (20 U/ml PHA P, DIFCO, Detroit Mich.).

HIV-1 Infection of C8166 Cells

Samples of 3×10⁵/100 μL C8166 cells were preincubated in 96-well microtiter plates in culture medium containing various concentrations of peptide. After a 1 h treatment at 37° C., 100 μl of diluted viral solution of HIV-1 was added. The cells were exposed to the virus for 1 h at 37° C. at a multiplicity of infection of 1000 TCID₅₀ per ml. The cells were washed three times and cultured at 3×10⁵/ml of R10 with the treatment in 24-well plates incubated at 37° C. C8166 culture medium was replaced at Day-4 post-infection. The treatment was permanent before virus adsorption, during virus adsorption and after infection. Assays on C8166 cells have been performed at least twice and in duplicate. Toxicity was evaluated by daily cell count and trypan-blue exclusion assay. Infection of C8166 T-cells with HIV-1 was assessed by virus-induced cytopathic effects (syncytia formation) and by quantification of cell free p24 viral protein in the culture supernatants. Measurements of HIV-1 p24^{gag} concentration in the culture supernatants were achieved by ELISA (ALLIANCE® HIV-1 p24 kit, Perkin Elmer, life sciences, USA).

Infection of Human Peripheral Blood Lymphocytes (PBL)

Samples of 10⁶/100 μL PBL cells were preincubated in 96-well microtiter plates in culture medium containing various concentrations of peptides. After a 1 h treatment at 37° C., 100 μl of diluted viral solution of HIV-1 was added. The cells were exposed to the virus for 1 h at 37° C. at a multiplicity of infection of 1000 TCID₅₀ per ml. The cells were washed three times and cultured at 110⁶/ml of medium with the treatment in 24-well plates incubated at 37° in culture medium with the peptides in 5% CO2. The treatment was permanent before virus adsorption, during virus adsorption and after infection. The PBL culture medium was replaced every 3-4 days during three weeks always in the presence of peptide. The cell viability was assessed by cell counts and trypan-blue exclusion assay. The viral production in the culture supernatant was quantified by p24 ELISA test, as described earlier. All the experiments have been done in blind-tests. Tests have been done in duplicate.

Results

TABLE 1
Experiment on C8166 cells with HIV-1NL-4-3

| | | P 24 | | | | P 24 |
|---|---|---|---|---|---|---|
| | Day 4 | (pg/ml) | Day 5 | Day 6 | Day 7 | (pg/ml) |
| S1 | | | | | | |
| 5 μM | -TOX | NEG | -TOX | -TOX | -TOX | 72 |
| | -TOX | NEG | -TOX | -TOX | -TOX | 73 |
| 2 μM | − | 19 | − | − | − | NEG |
| | − | 46 | − | − | − | 223 |
| 1 μM | − | 14 | (+) | + | + | 188 |
| | − | 10 | − | − | − | 169 |
| 0.5 μM | − | 40 | − | + | + | 234 |
| | − | 45 | (+) | + | + | 75 |
| S2 | | | | | | |
| 5 μM | -TOX | NEG | -TOX | TOX | TOX | ND |
| | -TOX | NEG | -TOX | TOX | TOX | |
| 2 μM | − | NEG | − | − | − | NEG |
| | − | NEG | − | − | − | 24 |
| 1 μM | − | NEG | − | − | − | 181 |
| | − | NEG | − | − | − | 245 |
| 0.5 μM | − | NEG | − | − | + | 150 |
| | − | NEG | − | − | + | 73 |
| S3 | | | | | | |
| 5 μM | − | NEG | − | − | − | 28 |
| | − | NEG | − | − | − | 189 |
| 2 μM | − | NEG | − | − | − | 18 |
| | − | NEG | − | − | − | 7 |
| 1 μM | − | NEG | − | − | − | 9 |
| | − | 15 | − | − | + | 234 |
| 0.5 μM | − | NEG | − | − | + | 97 |
| | − | 7 | − | − | (+) | NEG |
| S4 | | | | | | |
| 5 μM | − | NEG | + | + | ++ | 21120 |
| | − | 12 | + | + | ++ | 15674 |
| 2 μM | − | 17 | + | + | ++ | 17872 |
| | − | 14 | + | + | ++ | 24806 |
| 1 μM | − | 96 | + | + | ++ | 19801 |
| | − | 244 | + | + | ++ | 21640 |
| 0.5 μM | − | 43 | + | + | ++ | 19801 |
| | − | 28 | + | + | ++ | 25000 |
| S5 | | | | | | |
| 10 μM | − | NEG | − | − | − | 81 |
| | − | NEG | − | − | − | 134 |
| 5 μM | − | NEG | − | − | − | 66 |
| | − | NEG | − | − | + | 71 |
| 2 μM | − | NEG | − | − | − | 206 |
| | − | NEG | − | − | − | 76 |
| 1 μM | − | NEG | − | − | − | NEG |
| | − | NEG | − | − | + | 152 |
| 0.5 μM | − | NEG | − | − | − | NEG |
| | − | NEG | − | − | + | 233 |
| S6 | | | | | | |
| 10 μM | − | NEG | − | − | − | NEG |
| | − | NEG | − | − | − | NEG |
| 5 μM | − | NEG | − | − | − | NEG |
| | − | NEG | − | − | − | NEG |
| 2 μM | − | NEG | − | − | − | 67 |
| | − | 8 | − | − | + | 164 |
| 1 μM | − | 12 | − | (+) | + | 218 |
| | − | 25 | − | − | + | 186 |
| 0.5 μM | − | 75 | − | + | + | 1417 |
| | − | 14 | − | + | ++ | 20139 |
| AZT | | | | | | |
| 1 μM | − | NEG | − | − | − | NEG |
| | − | NEG | − | − | − | NEG |
| SPC3 | | | | | | |
| 5 μM | − | NEG | − | − | − | NEG |
| | − | NEG | − | − | − | NEG |
| 2 μM | − | 17 | (+) | + | ++ | 7589 |
| | − | 6 | (+)− | + | ++ | 14787 |
| TCell | − | NEG | − | − | − | NEG |
| | − | NEG | − | − | − | NEG |
| NL4-3 1/1000 | (+) | 244 | + | ++ | ++ | 25000 |
| | (+) | 244 | + | ++ | ++ | 25000 |

Key
S1: SPC3-(η-aminocaprylic acid)
S2: SPC3-Penetratin
S3: SPC3-Tat
S4 SPC3-(ε-aminocaproic acid)
S5: SPC3-(δ-aminovaleric acid
S6: SPC3-(γ-aminobutyric acid)
++, +, (+), (+)− and − represent decreasing numbers of syncitia formed

TABLE 2
Antiviral Activity Experiment on C8166 cells with HIV NL-4-3

| | | P 24 | | | | P 24 |
|---|---|---|---|---|---|---|
| | Day 4 | (pg/ml) | Day 5 | Day 6 | Day 7 | (pg/ml) |
| S2 | | | | | | |
| 1 μM | − | NEG | − | − | − | 67 |
| | − | NEG | − | − | − | 19 |
| 0.5 μM | − | 2 | − | − | + | 91 |
| | − | 2 | − | − | (+) | 115 |
| 0.1 μM | (+) | 75 | + | + | ++ | 596 |
| | (+) | 7 | + | + | ++ | 113 |
| 0.05 μM | (+) | 143 | + | ++ | ++ | 2923 |
| | (+) | 28 | ++ | ++ | ++ | 468 |
| S3 | | | | | | |
| 1 μM | − | 3 | − | − | − | 327 |
| | − | 1 | − | − | − | 746 |
| 0.5 μM | − | 14 | − | − | (+) | 189 |
| | − | 2 | − | − | + | 72 |
| 0.1 μM | (+) | 61 | − | + | ++ | 787 |
| | (+) | 33 | + | + | ++ | 496 |
| 0.05 μM | (+) | 261 | + | ++ | ++ | 3664 |
| | (+) | 94 | + | ++ | ++ | 2064 |
| S5 | | | | | | |
| 1 μM | − | 44 | − | − | − | 39 |
| | − | 18 | − | − | (+) | 385 |
| 0.5 μM | − | 9 | − | (+) | + | 39 |
| | − | 58 | − | − | + | 72 |
| 0.1 μM | (+) | 8 | + | ++ | ++ | 435 |
| | (+) | 73 | + | ++ | ++ | 137 |
| 0.05 μM | (+) | 66 | + | ++ | ++ | 3185 |
| | (+) | 33 | + | ++ | ++ | 2159 |
| S6 | | | | | | |
| 1 μM | − | 36 | − | − | − | 54 |
| | − | 17 | − | − | − | 1668 |
| 0.5 μM | − | 14 | − | (+) | + | 493 |
| | − | 54 | − | − | + | 288 |
| 0.1 μM | (+) | 90 | + | + | ++ | 1957 |
| | (+) | 14 | + | ++ | ++ | 613 |
| 0.05 μM | (+) | 21 | + | ++ | ++ | 1271 |
| | (+) | 303 | + | ++ | ++ | 1022 |
| S7 | | | | | | |
| 5 μM | − | NEG | − | − | + | 7 |
| | − | NEG | − | + | ++ | 105 |
| 1 μM | (+) | 44 | + | ++ | ++ | 7191 |
| | (+) | 19 | + | ++ | ++ | 641 |
| AZT | | | | | | |
| 1 μM | − | NEG | − | − | − | 1 |
| | − | NEG | − | − | − | NEG |
| SPC3 | | | | | | |
| 5 μM | − | NEG | (+) | + | ++ | 288 |
| | − | NEG | (+) | + | ++ | 342 |
| 1 μM | (+) | 35 | + | ++ | ++ | 3943 |
| | (+) | 11 | + | ++ | ++ | 1297 |
| TCell | − | NEG | − | − | − | NEG |
| | − | NEG | − | − | − | NEG |
| NL4-3 1/1000 | (+) | 303 | ++ | ++ | ++ | 25000 |
| | (+) | 184 | ++ | ++ | ++ | 25000 |

S1-S6 as above, S7: SPC3-Kpam
++, +, (+), (+)− and − represent decreasing numbers of syncitia formed.

All tested analogues showed an increased activity as compared to SPC3 (between 5 and 150 fold).

Similar results were obtained on PBL:

| IC₁₀₀ | S1 | S2 | S3 | S4 | S5 | S6 | SPC3 |
|---|---|---|---|---|---|---|---|
| | 0.1 μM | 0.01 | 0.1 μM | 0.5 μM | 0.01 μM | 0.01 μM | 2 μM |
| | | μM | | | | | |

The best agents were S5 and S6, SPC3-(δ-aminovaleric acid) and SPC3-(γ-aminobutyric acid) respectively, with an IC₅₀ of 0.1 to 0.01 μM and no toxicity on cells at doses up to 10 μM.

TABLE 3
Antiviral Activity Experiment on C8166 cells with HIV-1 subtype B NL 4-3

| Name | Formula | IC₅₀ (μM) |
|---|---|---|
| SPC3 | (GPGRAF)₈-K₄—K₂—K—NHCH₂CH₂COOH | 0.5 |
| SPC3 dimer valeric acid | (GPGRAF)₂-K—NHCH₂CH₂CH₂CH₂COOH | 0.05 |
| SPC3 monomer | GPGRAF | >10 |
| SPC3 monomer valeric acid | GPGRAF-NHCH₂CH₂CH₂CH₂COOH | 0.02 |
| RL | (RQGYSPL)₈-K₄—K₂—K—NHCH₂CH₂COOH | 0.01 |
| RL dimer | (RQGYSPL)₂-K—NHCH₂CH₂COOH | 0.02 |
| RL monomer | RQGYSPL | 0.5 |
| RL dimer valeric acid | (RQGYSPL)₂-K—NHCH₂CH₂CH₂CH₂COOH | 0.05 |
| RL monomer valeric acid | RQGYSPL-NHCH₂CH₂CH₂CH₂COOH | 0.05 |
| RS | (RQGYS)₂-K—NHCH₂CH₂COOH | 0.1 |
| RS monomer | RQGYS | 0.2 |
| RS dimer valeric acid | (RQGYS)₂-K—NHCH₂CH₂CH₂CH₂COOH | 0.05 |
| RS monomer valeric acid | RQGYS-NHCH₂CH₂CH₂CH₂COOH | 0.2 |

The above table shows that the graft of a valeric acid root on monomers of the peptides RL and SPC3 increases their activity on C8166 cells. In the case of SPC3, the activity becomes greater than that of the original polymerized peptide.

TABLE 4
Experiment on PBL with NL 4-3 strain

| | | IC₅₀ | IC₁₀₀ |
|---|---|---|---|
| Name | Formula | (μM) | (μM) |
| SPC3 | (GPGRAF)₈-K₄—K₂—K—NHCH₂CH₂COOH | 0.01 | 0.1 |
| SPC3 monomer valeric acid | GPGRAF-NHCH₂CH₂CH₂CH₂COOH | 0.02 | 0.1 |
| RL | (RQGYSPL)₈-K₄—K₂—K—NHCH₂CH₂COOH | 0.005 | 0.1 |
| RL dimer | (RQGYSPL)₂-K—NHCH₂CH₂COOH | 0.01 | 0.1 |
| RL dimer valeric acid | (RQGYSPL)₂-K—NHCH₂CH₂CH₂CH₂COOH | 0.005 | 0.05 |
| RL monomer valeric acid | RQGYSPL-NHCH₂CH₂CH₂CH₂COOH | 0.01 | 1 |

The results show that monomers or dimers of the original peptides have an activity comparable to that of the octomers. SPC3 monomer valeric acid has an IC₁₀₀ of 0.1 μM, as compared to 2 μm for normal SPC3, and 0.5 μM for SPC3 valeric acid. This is of importance as SPC3 contains 56 amino-acid residues, whereas the monomer contains only 6.

TABLE 5
Experiment on PBMC with HIV-1 89.6 subtype B dualtropic (X4R5)

| | | IC₅₀ | IC₁₀₀ |
|---|---|---|---|
| Name | Formula | (μM) | (μM) |
| SPC3 | (GPGRAF)₈-K₄—K₂—K—NHCH₂CH₂COOH | 0.06 | 0.5 |
| SPC3 dimer valeric acid | (GPGRAF)₂-K—NHCH₂CH₂CH₂CH₂COOH | 0.008 | 0.5 |
| SPC3 monomer valeric acid | GPGRAF-NHCH₂CH₂CH₂CH₂COOH | 0.01 | 0.5 |
| RL | (RQGYSPL)₈-K₄—K₂—K—NHCH₂CH₂COOH | 0.006 | 0.05 |
| RL dimer valeric acid | (RQGYSPL)₂-K—NHCH₂CH₂CH₂CH₂COOH | 0.01 | 0.5 |
| RL monomer valeric acid | RQGYSPL-NHCH₂CH₂CH₂CH₂COOH | 0.01 | 0.1 |

TABLE 6
Antiviral Activity Experiment on C8166 cells with HIV NL-4-3

| | Day | P 24 | | | | P 24 |
|---|---|---|---|---|---|---|
| | 4 | (pg/ml) | Day 5 | Day 6 | Day 7 | (pg/ml) |
| GPG | | | | | | |
| 5 μM | − | | − | − | − | 1 |
| | − | | − | − | − | 5 |
| 1 μM | − | | − | − | − | 3.8 |
| | − | | − | − | − | 5.4 |
| 0.5 μM | − | | − | − | − | 7.9 |
| | − | | − | − | − | 18 |
| 0.1 μM | − | | − | − | + | 525 |
| | − | | − | − | + | 5764 |
| 0.05 μM | − | | − | (+) | + | 7330 |
| | − | | − | (+) | + | 9810 |
| 0.01 μM | − | | (+) | + | ++ | 13850 |
| | − | | (+) | + | ++ | 11756 |
| 0.005 μM | − | | + | ++ | ++/T | 23810 |
| | − | | + | ++ | ++/T | 23810 |
| GPG | | | | | | |
| valeric acid | | | | | | |
| 5 μM | − | | − | − | − | 5.6 |
| | − | | − | − | − | 3.2 |
| 1 μM | − | | − | − | − | 5.636 |
| | − | | − | − | − | 4.8 |
| 0.5 μM | − | | − | − | − | 3.5 |
| | − | | − | − | − | 5.6 |
| 0.1 μM | − | | − | (+) | + | 126 |
| | − | | − | (+) | + | 3810 |
| 0.05 μM | − | | − | (+) | + | 1850 |
| | − | | − | (+) | + | 9867 |
| 0.01 μM | − | | + | + | ++ | 11810 |
| | − | | + | + | ++ | 13740 |
| 0.005 μM | − | | + | ++ | ++/T | 23810 |
| | − | | + | ++ | ++/T | 23810 |
| GPGR | | | | | | |
| 5 μM | − | | − | − | − | 9.425 |
| | − | | − | − | − | 3.375 |
| 1 μM | − | | − | − | + | 1103 |
| | − | | − | − | + | 485 |
| 0.5 μM | − | | − | − | + | 2507 |
| | − | | − | − | + | 2840 |
| 0.1 μM | − | | (+) | + | + | 5810 |
| | − | | (+) | + | + | 10110 |
| 0.05 μM | − | | + | + | ++ | 2507 |
| | − | | + | + | ++ | 13870 |
| 0.01 μM | − | | + | ++ | ++ | 23810 |
| | − | | + | ++ | ++ | 23810 |
| 0.005 μM | − | | ++ | ++ | ++/T | 23810 |
| | − | | ++ | ++ | ++/T | 23810 |
| GPGR | | | | | | |
| valeric acid | | | | | | |
| 5 μM | − | | − | − | − | 2.36 |
| | − | | − | − | − | 2.4 |
| 1 μM | − | | − | − | + | 104 |
| | − | | − | − | + | 179 |
| 0.5 μM | − | | − | − | + | 105 |
| | − | | − | − | + | 510 |
| 0.1 μM | − | | (+) | + | + | 433 |
| | − | | (+) | + | + | 507 |
| 0.05 μM | − | | (+) | + | ++ | 9840 |
| | − | | (+) | + | ++ | 11830 |
| 0.01 μM | − | | + | ++ | ++ | 21800 |
| | − | | + | ++ | ++ | 23810 |
| 0.005 μM | − | | + | ++ | ++ | 23810 |
| | − | | + | ++ | ++ | 23810 |
| GPGRA | | | | | | |
| 5 μM | − | | − | − | − | 3.62 |
| | − | | − | − | − | 13 |
| 1 μM | − | | − | − | − | 2.9 |
| | − | | − | − | − | 3.2 |
| 0.5 μM | − | | − | − | − | 2.1 |
| | − | | − | − | − | 2.1 |
| 0.1 μM | − | | (+) | + | + | 2838 |
| | − | | (+) | + | + | 2435 |
| 0.05 μM | − | | (+) | + | ++ | 4230 |
| | − | | (+) | + | ++ | 8910 |
| 0.01 μM | − | | + | ++ | ++/T | 15650 |
| | − | | + | ++ | ++/T | 16810 |
| 0.005 μM | − | | + | ++ | ++/T | 23810 |
| | − | | + | ++ | ++/T | 23810 |
| GPGRA | | | | | | |
| valeric acid | | | | | | |
| 5 μM | − | | − | − | − | 2.7 |
| | − | | − | − | − | 1.8 |
| 1 μM | − | | − | − | − | 2.3 |
| | − | | − | − | − | 1.9 |
| 0.5 μM | − | | − | − | − | 2 |
| | − | | − | − | − | 2.2 |
| 0.1 μM | − | | (+) | + | + | 2352 |
| | − | | (+) | + | + | 1011 |
| 0.05 μM | − | | (+) | + | + | 6830 |
| | − | | (+) | + | + | 3820 |
| 0.01 μM | − | | + | ++ | ++ | 13030 |
| | − | | + | ++ | ++ | 13810 |
| 0.005 μM | − | | + | ++ | ++/T | 23810 |
| | − | | + | ++ | ++/T | 23810 |
| SPC3 | | | | | | |
| monomer | | | | | | |
| valeric acid | | | | | | |
| 5 μM | − | | − | − | − | 3 |
| | − | | − | − | − | 3 |
| 1 μM | − | | − | − | (+) | 325 |
| | − | | − | − | (+) | 445 |
| 0.5 μM | − | | − | (+) | + | 1840 |
| | − | | − | (+) | + | 2830 |
| 0.1 μM | − | | (+) | + | ++ | 11810 |
| | − | | (+) | ++ | ++ | 1507 |
| 0.05 μM | − | | + | ++ | ++ | 3810 |
| | − | | + | ++ | ++ | 21810 |
| 0.01 μM | − | | + | ++ | ++/T | 21810 |
| | − | | + | ++ | ++/T | 21810 |
| 0.005 μM | − | | + | ++ | ++/T | 23810 |
| | − | | + | ++ | ++/T | 23810 |
| SPC3 | | | | | | |
| 5 μM | − | | − | − | − | 3 |
| | − | | − | − | − | 3 |
| 1 μM | − | | − | − | (+) | 1692 |
| | − | | − | − | (+) | 776 |
| 0.5 μM | − | | − | (+) | + | 5173 |
| | − | | − | (+) | + | 4840 |
| 0.1 μM | − | | (+) | + | ++ | 17810 |
| | − | | (+) | ++ | ++ | 19850 |
| 0.05 μM | − | | + | ++ | ++/T | 23810 |
| | − | | + | ++ | ++/T | 23810 |
| 0.01 μM | − | | + | ++ | ++/T | 23810 |
| | − | | + | ++ | ++/T | 23810 |
| 0.005 μM | − | | + | ++ | ++/T | 23810 |
| | − | | + | ++ | ++/T | 23810 |
| TCell | − | | − | − | − | 3 |
| | − | | − | − | − | 3 |
| NL4-3 1/1000 | (+) | | + | ++ | ++/T | 23810 |
| | (+) | | + | ++ | ++/T | 23810 |

TABLE 7
Antiviral Activity Experiment on C8166 cells with HIV NL-4-3

| | | P 24 | | | | P 24 |
|---|---|---|---|---|---|---|
| | Day 4 | (pg/ml) | Day 5 | Day 6 | Day 7 | (pg/ml) |
| GPG | | | | | | |
| 5 μM | − | | − | − | − | 2 |
| | − | | − | − | − | 79 |
| 1 μM | − | | − | − | (+) | 42 |
| | − | | − | − | (+) | 62 |
| 0.5 μM | − | | − | − | (+) | 126 |
| | − | | − | − | (+) | 165 |
| 0.1 μM | − | | − | (+) | + | 807 |
| | − | | − | (+) | + | 1506 |
| 0.5 μM | − | | − | (+) | + | 1810 |
| | − | | − | (+) | + | 3810 |
| 0.01 μM | (+) | | (+) | + | ++ | 9810 |
| | (+) | | (+) | + | ++ | 15810 |
| GPG | | | | | | |
| valeric acid | | | | | | |
| 5 μM | − | | − | − | − | 60 |
| | − | | − | − | − | 34 |
| 1 μM | − | | − | − | − | 86 |
| | − | | − | − | − | 74 |
| 0.5 μM | − | | − | − | (+) | 126 |
| | − | | − | − | (+) | 44 |
| 0.1 μM | − | | − | (+) | + | 108 |
| | − | | − | (+) | + | 130 |
| 0.05 μM | − | | − | (+) | + | 3810 |
| | − | | − | (+) | + | 2300 |
| 0.01 μM | − | | (+) | + | ++ | 3800 |
| | − | | (+) | + | ++ | 23000 |
| GPGR | | | | | | |
| 5 μM | − | | − | − | (+) | 152 |
| | − | | − | − | (+) | 152 |
| 1 μM | − | | − | − | (+) | 316 |
| | − | | − | − | (+) | 343 |
| 0.5 μM | − | | − | − | + | 23000 |
| | − | | − | − | + | 15810 |
| 0.1 μM | − | | (+) | + | + | 5810 |
| | − | | (+) | + | + | 23000 |
| 0.05 μM | (+) | | + | + | ++ | 13810 |
| | (+) | | + | + | ++ | 12980 |
| 0.01 μM | (+) | | + | ++ | ++ | 23810 |
| | (+) | | + | ++ | ++ | 23810 |
| GPGR | | | | | | |
| valeric acid | | | | | | |
| 5 μM | − | | − | − | − | 2 |
| | − | | − | − | − | 2 |
| 1 μM | − | | − | − | − | 53 |
| | − | | − | − | − | 64 |
| 0.5 μM | − | | − | − | + | 2740 |
| | − | | − | − | + | 2840 |
| 0.1 μM | − | | (+) | + | + | 2173 |
| | − | | (+) | + | + | 9810 |
| 0.05 μM | − | | (+) | + | ++ | 9860 |
| | − | | (+) | + | ++ | 17800 |
| 0.01 μM | − | | + | ++ | ++/T | 3800 |
| | − | | + | ++ | ++ | 21300 |
| GPGRA | | | | | | |
| 5 μM | − | | − | − | (+) | 99 |
| | − | | − | − | (+) | 100 |
| 1 μM | − | | − | − | (+) | 117 |
| | − | | − | − | (+) | 119 |
| 0.5 μM | − | | − | − | + | 2070 |
| | − | | − | − | + | 5410 |
| 0.1 μM | − | | (+) | + | + | 2837 |
| | − | | (+) | + | ++ | 9310 |
| 0.05 μM | − | | (+) | + | ++ | 4230 |
| | − | | (+) | + | ++ | 8910 |
| 0.01 μM | − | | + | ++ | ++/T | 15650 |
| | − | | + | ++ | ++/T | 16810 |
| GPGRA | | | | | | |
| valeric acid | | | | | | |
| 5 μM | − | | − | − | − | 2.7 |
| | − | | − | − | − | 3 |
| 1 μM | − | | − | − | − | 13 |
| | − | | − | − | − | 10 |
| 0.5 μM | − | | − | − | (+) | 234 |
| | − | | − | − | (+) | 576 |
| 0.1 μM | − | | − | (+) | + | 2356 |
| | − | | − | (+) | + | 2416 |
| 0.05 μM | − | | (+) | + | + | 3810 |
| | − | | (+) | + | + | 11820 |
| 0.01 μM | − | | + | ++ | ++ | 13870 |
| | − | | + | ++ | ++ | 11810 |
| TCell | − | | − | − | − | 2 |
| | − | | − | − | − | 6 |
| NL4-3 1/1000 | (+) | | + | ++ | ++/T | 23810 |
| | (+) | | + | ++ | ++/T | 15670 |
| | | | | | ++/T | 19750 |

TABLE 8
Antiviral Activity Experiment on C8166 cells with HIV 1 NDK

| | | P 24 | | | | P 24 |
|---|---|---|---|---|---|---|
| | Day 4 | (pg/ml) | Day 5 | Day 6 | Day 7 | (pg/ml) |
| GPG | | | | | | |
| 5 μM | − | | − | − | + | 2733 |
| | − | | − | − | + | 2400 |
| 1 μM | − | | (+) | + | + | 2507 |
| | − | | (+) | + | + | 3810 |
| 0.5 μM | − | | + | ++ | ++ | 21110 |
| | − | | + | ++ | ++ | 23810 |
| 0.1 μM | − | | + | ++ | ++ | 23810 |
| | − | | + | ++ | ++ | 23810 |
| 0.05 μM | (+) | | + | ++ | ++ | 23810 |
| | (+) | | + | ++ | ++ | 23810 |
| 0.01 μM | + | | + | ++ | ++ | 23810 |
| | + | | + | ++ | ++ | 23810 |
| 0.005 μM | + | | + | ++ | ++ | 23810 |
| | + | | + | ++ | ++ | 23810 |
| GPG | | | | | | |
| valeric acid | | | | | | |
| 5 μM | − | | − | − | (+) | 284 |
| | − | | − | − | (+) | 217 |
| 1 μM | − | | − | − | + | 2810 |
| | − | | − | − | + | 1840 |
| 0.5 μM | − | | − | + | ++ | 2578 |
| | − | | − | + | ++ | 3140 |
| 0.1 μM | − | | − | + | ++ | 3507 |
| | − | | − | + | ++ | 3670 |
| 0.05 μM | − | | (+) | ++ | ++ | 11810 |
| | − | | (+) | ++ | ++ | 15879 |
| 0.01 μM | (+) | | + | ++ | ++ | 23810 |
| | (+) | | + | ++ | ++ | 23810 |
| 0.005 μM | (+) | | + | ++ | ++ | 23810 |
| | (+) | | + | ++ | ++ | 23810 |
| GPGR | | | | | | |
| 5 μM | − | | − | + | ++ | 2840 |
| | − | | − | + | ++ | 7810 |
| 1 μM | − | | − | + | ++ | 9870 |
| | − | | − | + | ++ | 13890 |
| 0.5 μM | − | | (+) | ++ | ++ | 9810 |
| | − | | (+) | ++ | ++ | 15856 |
| 0.1 μM | − | | (+) | ++ | ++ | 21810 |
| | − | | (+) | ++ | ++ | 23870 |
| 0.05 μM | − | | + | ++ | ++ | 23810 |
| | − | | + | ++ | ++ | 23810 |
| 0.01 μM | − | | + | ++ | ++ | 23810 |
| | − | | + | ++ | ++ | 23810 |
| 0.005 μM | − | | + | ++ | ++/T | 23810 |
| | − | | + | ++ | ++/T | 23810 |
| GPGR | | | | | | |
| valeric acid | | | | | | |
| 5 μM | − | | − | (+) | + | 3810 |
| | − | | − | (+) | + | 3810 |
| 1 μM | − | | − | + | ++ | 2840 |
| | − | | − | + | ++ | 3810 |
| 0.5 μM | − | | − | + | ++ | 7810 |
| | − | | − | + | ++ | 3840 |
| 0.1 μM | − | | (+) | ++ | ++ | 17890 |
| | − | | (+) | ++ | ++ | 23810 |
| 0.05 μM | − | | (+) | ++ | ++ | 23810 |
| | − | | (+) | ++ | ++ | 23810 |
| 0.01 μM | (+) | | + | ++ | ++ | 23810 |
| | (+) | | + | ++ | ++ | 23810 |
| 0.005 μM | (+) | | + | ++ | ++ | 23810 |
| | (+) | | + | ++ | ++ | 23810 |
| GPGRA | | | | | | |
| 5 μM | − | | − | − | + | 2726 |
| | − | | − | − | + | 2070 |
| 1 μM | − | | − | + | ++ | 3070 |
| | − | | − | + | ++ | 2403 |
| 0.5 μM | − | | − | ++ | ++ | 2070 |
| | − | | − | ++ | ++ | 5420 |
| 0.1 μM | − | | (+) | ++ | ++ | 13840 |
| | − | | (+) | ++ | ++ | 9310 |
| 0.05 μM | − | | (+) | ++ | ++ | 13010 |
| | − | | (+) | ++ | ++ | 10910 |
| 0.01 μM | (+) | | + | ++ | ++ | 15650 |
| | (+) | | + | ++ | ++ | 16810 |
| 0.005 μM | (+) | | + | ++ | ++ | 23810 |
| | (+) | | + | ++ | ++ | 23810 |
| GPGRA | | | | | | |
| valeric acid | | | | | | |
| 5 μM | − | | − | − | − | 32 |
| | − | | − | − | (+) | 108 |
| 1 μM | − | | − | − | + | 2000 |
| | − | | − | − | + | 2403 |
| 0.5 μM | − | | − | + | ++ | 3810 |
| | − | | − | + | ++ | 7810 |
| 0.1 μM | − | | (+) | ++ | ++ | 5600 |
| | − | | (+) | ++ | ++ | 6400 |
| 0.05 μM | − | | (+) | ++ | ++ | 3810 |
| | − | | (+) | ++ | ++ | 11789 |
| 0.01 μM | − | | + | ++ | ++ | 13810 |
| | − | | + | ++ | ++ | 18710 |
| 0.005 μM | (+) | | + | ++ | ++ | 23810 |
| | (+) | | + | ++ | ++ | 23810 |
| SPC3 | | | | | | |
| monomer | | | | | | |
| valeric acid | | | | | | |
| 5 μM | − | | − | − | − | 123 |
| | − | | − | − | − | 345 |
| 1 μM | − | | − | − | (+) | 1325 |
| | − | | − | − | (+) | 4345 |
| 0.5 μM | − | | − | + | ++ | 11840 |
| | − | | − | + | ++ | 12240 |
| 0.1 μM | − | | + | ++ | ++ | 11810 |
| | − | | + | ++ | ++ | 15307 |
| 0.05 μM | − | | + | ++ | ++ | 23810 |
| | − | | + | ++ | ++ | 21810 |
| 0.01 μM | − | | + | ++ | ++/T | 21810 |
| | − | | + | ++ | ++/T | 21810 |
| 0.005 μM | − | | + | ++ | ++/T | 23810 |
| | − | | + | ++ | ++/T | 23810 |
| SPC3 | | | | | | |
| 5 μM | − | | − | − | − | 12 |
| | − | | − | − | − | 240 |
| 1 μM | − | | − | − | (+) | 1692 |
| | − | | − | − | (+) | 3776 |
| 0.5 μM | − | | − | + | ++ | 15173 |
| | − | | − | + | ++ | 12840 |
| 0.1 μM | − | | (+) | + | ++ | 18810 |
| | − | | (+) | ++ | ++ | 20850 |
| 0.05 μM | − | | + | ++ | ++/T | 23810 |
| | − | | + | ++ | ++/T | 23810 |
| 0.01 μM | − | | + | ++ | ++/T | 23810 |
| | − | | + | ++ | ++/T | 23810 |
| 0.005 μM | − | | + | ++ | ++/T | 23810 |
| | − | | + | ++ | ++/T | 23810 |
| TCell | − | | − | − | − | |
| | − | | − | − | − | |
| NL4-3 1/1000 | (+) | | ++ | ++ | ++/T | 19657 |
| | (+) | | ++ | ++ | ++/T | 23810 |

TABLE 9
Antiviral Activity Experiment on C8166 cells with HIV-1 subtype B NL 4-3

| | | IC₅₀ | IC₁₀₀ |
|---|---|---|---|
| Name | Formula | (μM) | (μM) |
| GPG | GPG | 0.01 | 5 |
| | | 0.01 | 5 |
| GPG valeric acid | GPG-NHCH₂CH₂CH₂CH₂COOH | 0.01 | 0.5 |
| | | 0.01 | 1 |
| GPGR | GPGR | 0.06 | 5 |
| | | 0.1 | >5 |
| GPGR valeric acid | GPGR-NHCH₂CH₂CH₂CH₂COOH | 0.03 | 5 |
| | | 0.01 | 1 |
| GPGRA | GPGRA | 0.03 | 0.5 |
| | | 0.02 | >5 |
| GPGRA valeric acid | GPGRA-NHCH₂CH₂CH₂CH₂COOH | 0.01 | 0.1 |
| | | 0.01 | 1 |
| SPC3 monomer valeric acid | GPGRAF-NHCH₂CH₂CH₂CH₂COOH | 0.05 | 5 |
| SPC3 | (GPGRAF)₈-K₄—K₂—K—NHCH₂CH₂COOH | 0.5 | 5 |

TABLE 10
Antiviral Activity Experiment on C8166 cells with HIV 1 NDK

| | | IC₅₀ | IC₁₀₀ |
|---|---|---|---|
| Name | Formula | (μM) | (μM) |
| GPG | GPG | 0.5 | >5 |
| GPG valeric acid | GPG-NHCH₂CH₂CH₂CH₂COOH | 0.02 | 5 |
| GPGR | GPGR | 0.5 | >5 |
| GPGR valeric acid | GPGR-NHCH₂CH₂CH₂CH₂COOH | 0.3 | >5 |
| GPGRA | GPGRA | 0.04 | >5 |
| GPGRA valeric acid | GPGRA-NHCH₂CH₂CH₂CH₂COOH | >5 | 5 |
| SPC3 monomer valeric acid | GPGRAF-NHCH₂CH₂CH₂CH₂COOH | 0.2 | 5 |
| SPC3 | (GPGRAF)₈-K₄—K₂—K—NHCH₂CH₂COOH | 0.6 | 5 |

1. A compound comprising a water soluble antiviral peptide including one of the sequences GPG and RQGY and, bonded to the C-end of the peptide, a terminator which is either (a) an ω-amino-fatty acid having from 4 to 10 carbon atoms and from 0 to 2 carbon-carbon double bonds or (b) a peptidic cell membrane penetrating agent.

2. A compound according to claim 1 in which the peptide is a multiple branch peptide construction (MBPC), each branch of which contains the peptide sequence GPG and the core of which is formed from lysine residues, and the terminator is bonded to the root lysine residue.

3. A compound according to claim 2 in which each branch of the MBPC is a peptide GPGRAF.

4. A compound according to claim 1 in which the peptide is a multiple branch peptide construction (MBPC), each branch of which contains the peptide sequence RQGY and the core of which is formed from lysine residues, and the terminator is bonded to the root lysine residue.

5. A compound according to claim 4 in which each branch of the MBPC is a peptide RQGYSPL.

6. A compound according to claim 4 in which each branch of the MBPC is a peptide RQGYS.

7. A compound according to claim 3 in which the MBPC has two branches.

8. A compound according to claim 3 in which the MBPC has eight branches.

9. A compound according to claim 1 in which the peptide is GPG, GPGR, GPGRA or GPGRAF.

10. A compound according to claim 1 in which the peptide is RQGYS or RQGYSPL.

11. A compound according to claim 1 in which the terminator is an ω-amino saturated fatty acid having from 4 to 8 carbon atoms.

12. A compound according to claim 1 in which the terminator is an ω-amino saturated fatty acid having from 4 to 6 carbon atoms.

13. A compound according to claim 1 in which the terminator is γ-aminobutyric acid, δ-aminovaleric acid or ε-aminocaproic acid.

14. A compound according to claim 1 in which the terminator is a TAT-derived peptide, penetratin® or Kpam.

15. A compound according to claim 5 in which the MBPC has two branches.

16. A compound according to claim 6 in which the MBPC has two branches

17. A compound according to claim 5 in which the MBPC has eight branches.

18. A compound according to claim 6 in which the MBPC has eight branches.
